SEQUENCE LISTING (19 sequences)

SEQ ID NO: 1 - protein, 665 aa, Zea mays L.
MATMSAASHRCCASSLRALTVLFVLAALVSDVGGRHHHHVCPPYFSCGGFSNISYPFRRQGDPSGCGVQSYELVCTDTDATIRIGSGTYTVLSINSTYSYFWVVDADLDIQSSCPLPWWDHHGETSTANSYRRRTEFRPYFLYPNSMSIIFVNCSKPIENNDIYEPVPCLSNSSFIYLLTHYSYGYALAEILEPSCGYLAMIYLGGPGIPVPKNTSYPDVVKLMRNGFGLRFPSSIGDRGIRECFAESVRNFLKEPRKYQIVDILMVEELWSCFLDQHGSTNNVVTSVIIDIIKTIPICMWLLKSTHVFCRLVLMPLAVFVFLAHKYWKARITIDAVEKFLRMQQMLVPMRYAYTNIIAITGHFREKLGQGGYGSVYKGVLQPGEVHVAVKMLGNSNCNGEEFISEVATIGKIHHFNVVRLIGFCSEENRRALIYEFMPHGSLDKYIFSSEKSFSWDKLNEIALGIARGLNYLHHGCDMQIVHFDIKPHNILLDSNFVPKVADFGLAKLFPRDDSFVPLSATRGTIGYIAPEMVSRSFGVISSKSDVYSFGMLLLEMTGGRRNADPYAGSSSQAYYPSLVYSQLSQGDLGEISDGVDMHELEKKLCIIGLWCIQMKPQDRPTMSDVIEMLEVGVDGIQMPPRPFFCDDEGDSSYSAISESDTIEE

SEQ ID NO: 2 - DNA, 2238 bp, Zea mays L.
  source 1..2238: mol_type = genomic DNA
ggcaagaaggtccacgaatcacgcgagccaatcagtggcgtgcagtggcgactgcaaagataagtgggtagaaacaagagtcacccatggcgacgatgtctgcagcgtctcatcgctgctgtgcttcttccttgagagctttaacggtgttatttgtgttggcagctcttgtttcagatgttggcgggcgacatcatcatcatgtttgtcctccttatttctcctgcggtggttttagcaatatatcgtatccattccgtcggcaaggtgatccatcggggtgcggtgtccaatcgtatgagctggtttgcacggatacagacgctaccattcgcatcggcagtggaacgtataccgtgcttagcatcaactccacctattcttacttctgggtcgttgatgccgacctggacatccagagcagttgcccccttccctggtgggatcaccatggtgagaccagtactgccaactcatatcgtaggaggactgagttcaggccttatttcctttatccgaattcgatgtcgattatctttgtgaattgctcgaagccaatagagaacaatgatatatatgagccggtgccttgcttgagcaattcttctttcatctacttgctaactcactactcgtatggctatgctcttgctgagattctggagccctcatgcggttacctagccatgatttatttgggtggtccaggcataccggtgcccaagaatacaagctatccagatgttgttaagttaatgaggaatggatttggccttagatttccttcttcgattggtgaccgcggcatcagagaatgtttcgcagagtctgtgcgtaatttccttaaagagccaagaaagtatcagattgtggacattctaatggtcgaggaattatggtcttgttttctcgatcaacatggatcaactaataatgttgtcacttctgttatcatcgacattatcaaaacaataccaatatgtatgtggcttctgaaatctacacatgttttttgcaggcttgtattgatgccgctagcagtatttgtcttcctagcccataaatactggaaagcaaggattacaatagatgcagtcgagaagttcctgcggatgcagcagatgctcgttccgatgagatatgcatacacaaacatcattgctatcaccggtcattttagagaaaagctcggacaaggaggctacggttctgtatacaagggggtgctacagccaggtgaagtacatgttgctgtcaagatgttaggcaactccaactgtaatggagaagagttcatcagtgaggtcgccaccattggcaagatccaccatttcaatgttgtgcgcctcattgggttttgctccgaggaaaatagaagggcacttatctacgagttcatgccccatggatctctcgataagtacatcttctcgtcggagaagagtttctcatgggacaaactcaatgagatcgctctgggcattgctagaggtctcaactacctacatcacgggtgcgatatgcaaattgtacacttcgacatcaagccacacaacatccttcttgacagcaactttgttccaaaagttgctgattttgggcttgccaaactgttcccaagagacgacagtttcgtgccactgagcgctacgcggggaacgataggctatatagctccagagatggtatctcgaagctttggtgtcatctctagcaaatctgatgtgtatagctttggaatgctactgttggagatgacgggcgggcgaaggaacgcagatccttatgcaggaagctccagtcaagcatactacccatccttggtgtacagccagctaagccaaggagatttgggcgagatcagtgacggtgttgatatgcacgagttagagaagaagctatgtatcattggactttggtgcatccagatgaagccgcaagatcgaccgacgatgagcgacgtcatagagatgcttgaagttggtgtcgatggcatccaaatgcctccaaggccattcttttgtgatgacgagggtgatagttcttactctgcaatctctgaatcggatacaatagaagagtagtagtagtaaaatacacttgtgtatatttgatcttaatgtattttagactactaagaatgcaaggttgtcataagctaaggtactggttgtatacttttattgagagtgtggtaatgtgttttcatttaaaatacctcaatgaaccttttttatc

SEQ ID NO: 3 - DNA, 2213 bp, Zea mays L.
  source 1..2213: mol_type = genomic DNA
atggcgacgatgtctgcagcgtctcatcgctgctgtgcttcttccttgagagctttaacggtgttatttgtgttggcagctcttgtttcagatgttggcgggcgacatcatcatcatgtttgtcctccttatttctcctgcggtggttttagcaatatatcgtatccattccgtcggcaaggtgatccatcggggtgcggtgtccaatcgtatgagctggtttgcacggatacagacgctaccattcgcatcggcagtggaacgtataccgtgcttagcatcaactccacctattcttacttctgggtcgttgatgccgacctggacatccagagcagttgcccccttccctggtgggatcaccatggtgagaccagtactgccaactcatatcgtaggaggactgagttcaggccttatttcctttatccgaattcgatgtcgattatctttgtgaattgctcgaagccaatagagaacaatgatatatatgagccggtgccttgcttgagcaattcttctttcatctacttgctaactcactactcgtatggctatgctcttgctgagattctggagccctcatgcggttacctagccatgatttatttgggtggtccaggcataccggtgcccaagaatacaagctatccagatgttgttaagttaatgaggaatggatttggccttagatttccttcttcgattggtgaccgcggcatcagagaatgtttcgcagagtctgtgcggtatctgatcatcctatcctattttcctcctatgcatgactttgtcatctgaaaaccgtccgttgcattcccttcgtaattcttttatatgctatggcatggtcttgcagtaatttccttaaagagccaagaaagtatcagattgtggacattctaatggtcgaggaattatggtcttgttttctcgatcaacatggatcaactaataatgttgtcacttctgttatcatcgacattatcaaaacaataccaatatgtatgtggcttctgaaatctacacatggtactctctccggctatcatcatttgttgaataaacatcgtatgttttgtggcttctgtttttttttaattcttcatgtttacaaccttggatttttttcggcagttttttgcaggcttgtattgatgccgctagcagtatttgtcttcctagcccataaatactggaaagcaaggattacaatagatgcagtcgagaagttcctgcggatgcagcagatgctcgttccgatgagatatgcatacacaaacatcattgctatcaccggtcattttagagaaaagctcggacaaggaggctacggttctgtatacaagggggtgctacagccaggtgaagtacatgttgctgtcaagatgttaggcaactccaactgtaatggagaagagttcatcagtgaggtcgccaccattggcaagatccaccatttcaatgttgtgcgcctcattgggttttgctccgaggaaaatagaagggcacttatctacgagttcatgccccatggatctctcgataagtacatcttctcgtcggagaagagtttctcatgggacaaactcaatgagatcgctctgggcattgctagaggtctcaactacctacatcacgggtgcgatatgcaaattgtacacttcgacatcaagccacacaacatccttcttgacagcaactttgttccaaaagttgctgattttgggcttgccaaactgttcccaagagacgacagtttcgtgccactgagcgctacgcggggaacgataggctatatagctccagagatggtatctcgaagctttggtgtcatctctagcaaatctgatgtgtatagctttggaatgctactgttggagatgacgggcgggcgaaggaacgcagatccttatgcaggaagctccagtcaagcatactacccatccttggtgtacagccagctaagccaaggagatttgggcgagatcagtgacggtgttgatatgcacgagttagagaagaagctatgtatcattggactttggtgcatccagatgaagccgcaagatcgaccgacgatgagcgacgtcatagagatgcttgaagttggtgtcgatggcatccaaatgcctccaaggccattcttttgtgatgacgagggtgatagttcttactctgcaatctctgaatcggatacaatagaagagtag

SEQ ID NO: 4 - DNA, 7238 bp, Zea mays L.
  source 1..7238: mol_type = genomic DNA
tccactcgtgatccctgatcaccaaaccagcccttagccaatcacctgcaacttgatcttcgcaaaagctagttgttagtcgattaggttcgcctttgactctcatttgttaatgatagttgggtcctgtctgactagcagatagtctacaaaatttttgatcatcttcagtgtatcccaaatagtagggtgaagatggggagtgtcactcatttatttgcgttttatgccttatggatatgtaaagtgtgtataaaatggtttagagagaattaaattgtttattttgctaatcatttttctggtgtcgggtaccaaaatgcacatgttttgttgtacatgtaagttctttatgactgatgtgtagtcatcactacgagtgcttgaaatagtgttgggagctaggaggaagtctatgtattctgtagcagtagcattatgcgacatcattaccataattacaaataaactaatcgtagccacgatgttcaatacattaactttttggtaacaaataaatgtaaaatggggaatgagaggttgcaatgtctactgatttctacgatttttcttatcagtgagtatgttggtagcttctttttctctttctaagtataacatctttgtcgttttaaattcatgagaatgattaaatataataggggaactttgaatatgttcatattcttatctaattgtgcaagttctatcaatcatataaattctaaaataggcatgtcttataactttgattcttttgaacaatttagtttatggaccaactatgtttgtatatgaacagatgacagacatagcaaaacttgtctacaatcttatatatcttttaaggtacataaatatctaattttagtgatgctaacaaaagtatagaataacaattacggggcgtttggatcccttcattttagaagaattagaattcactcaataaagtgacttatttagtttagaatttgacattccaccacttttcaaagttaggtataagcctatctcaaatttatgtggtggaggatgagaaatgattttattcattagtagaatttgtttctactctgtaacttacatgacactcttcgtctcactcctctatagtaaaaatatagcacataaatatctccaacatcttgctaataatagtatacaaatatattttgcataaaacagaattagcttaattgatatatgtcaaaattactattattagaatggaattcaattcgaatgatccaaacgaggcgttagtgtttttatcatattaattccgtagcaacgcacgagtatataactagtctttaataaggtgtatacaaactatagattgttcaaacaatttctatcattatgctctagctataatttaactttgcaaaacacaccttggtcataataattttatattatcattaatcaccgaaaaataggagggtctagatgctttcaacattcataggaaagcatctcatctaatgtttcatatactgtatatattgataagatgaaaagataaacacgaggcggaccatacactttatcatttagttccaaattaactgacactaaagttcttttcataacagggactgaagagcatttctctactagtagtggagcagatccaaatagactgaaatatgccgataaatctaccaagtatatcatatgagtatacttcggtttaataagacactgactgagcaaggatgccggccgcttcaaggtttgcacttgaaagtagcactccagatgatattggttactgcattgaaataccttttttaattacattttattccgtatatatccaagtttatactggtttgcggtggtaaaattatctcttcatctccggtcccatatccccatcgaaggccatttgcaaaccagaaaaagacgcaaaagaatgactaccaagtcagcggcatcacaattatgtggaccagtcatgtgcgattttgttccacaaatacagcgaagatgctgaggctgtgaccccgggtgactcttgtttctacccacttatctttgcagtcgccactgcacgccactgattggctcgcgtgattcgtggaccttcttgccatggcgacgatgtctgcagcgtctcatcgctgctgtgcttcttccttgagagctttaacggtgttatttgtgttggcagctcttgtttcagatgttggcgggcgacatcatcatcatgtttgtcctccttatttctcctgcggtggttttagcaatatatcgtatccattccgtcggcaaggtgatccatcggggtgcggtgtccaatcgtatgagctggtttgcacggatacagacgctaccattcgcatcggcagtggaacgtataccgtgcttagcatcaactccacctattcttacttctgggtcgttgatgccgacctggacatccagagcagttgcccccttccctggtgggatcaccatggtgagaccagtactgccaactcatatcgtaggaggactgagttcaggccttatttcctttatccgaattcgatgtcgattatctttgtgaattgctcgaagccaatagagaacaatgatatatatgagccggtgccttgcttgagcaattcttctttcatctacttgctaactcactactcgtatggctatgctcttgctgagattctggagccctcatgcggttacctagccatgatttatttgggtggtccaggcataccggtgcccaagaatacaagctatccagatgttgttaagttaatgaggaatggatttggccttagatttccttcttcgattggtgaccgcggcatcagagaatgtttcgcagagtctgtgcggtatctgatcatcctatcctattttcctcctatgcatgactttgtcatctgaaaaccgtccgttgcattcccttcgtaattcttttatatgctatggcatggtcttgcagtaatttccttaaagagccaagaaagtatcagattgtggacattctaatggtcgaggaattatggtcttgttttctcgatcaacatggatcaactaataatgttgtcacttctgttatcatcgacattatcaaaacaataccaatatgtatgtggcttctgaaatctacacatggtactctctccggctatcatcatttgttgaataaacatcgtatgttttgtggcttctgtttttttttaattcttcatgtttacaaccttggatttttttcggcagttttttgcaggcttgtattgatgccgctagcagtatttgtcttcctagcccataaatactggaaagcaaggattacaatagatgcagtcgagaagttcctgcggatgcagcagatgctcgttccgatgagatatgcatacacaaacatcattgctatcaccggtcattttagagaaaagctcggacaaggaggctacggttctgtatacaagggggtgctacagccaggtgaagtacatgttgctgtcaagatgttaggcaactccaactgtaatggagaagagttcatcagtgaggtcgccaccattggcaagatccaccatttcaatgttgtgcgcctcattgggttttgctccgaggaaaatagaagggcacttatctacgagttcatgccccatggatctctcgataagtacatcttctcgtcggagaagagtttctcatgggacaaactcaatgagatcgctctgggcattgctagaggtctcaactacctacatcacgggtgcgatatgcaaattgtacacttcgacatcaagccacacaacatccttcttgacagcaactttgttccaaaagttgctgattttgggcttgccaaactgttcccaagagacgacagtttcgtgccactgagcgctacgcggggaacgataggctatatagctccagagatggtatctcgaagctttggtgtcatctctagcaaatctgatgtgtatagctttggaatgctactgttggagatgacgggcgggcgaaggaacgcagatccttatgcaggaagctccagtcaagcatactacccatccttggtgtacagccagctaagccaaggagatttgggcgagatcagtgacggtgttgatatgcacgagttagagaagaagctatgtatcattggactttggtgcatccagatgaagccgcaagatcgaccgacgatgagcgacgtcatagagatgcttgaagttggtgtcgatggcatccaaatgcctccaaggccattcttttgtgatgacgagggtgatagttcttactctgcaatctctgaatcggatacaatagaagagtagtagtagtaaaatacacttgtgtatatttgatcttaatgtattttagactactaagaatgcaaggttgtcataagctaaggtactggttgtatacttttattgagagtgtggtaatgtgttttcatttaaaatacctcaatgaaccttttttatcaaaaactcctcaagaacattttgtttgccaacaaacataggatcacggtcccagccgttgtattcagctattgcacaagtttttaagttatgcataacaaattaagtgaactagcatattacccgcgctagcaccaagataatatttgacaatacaaattaaataaccaaaaggttattcatctcatataatataatgcccatgctaaagaccgctcatgtagaacgcgacattcatttgaaggcattagtaccagttgttgtagaatcgatattgattcaggcgtcagtaccgattttaatgaccacgtcctcatggatggcaatagtatagattggtgtctcaaatcaatgcaaaaggactctatctgagttagttaggtggtctgagtgacactcaaattctaaattctaagtttgagctcctatgaaagcaaattttaggctacggttaaaaattctcttgtatcattattcttgtgtattcaactgatgtcattattcttatttgtcttcactatagaatttgcatttcctacacacactacctatatgatttaaggttcaggatcgtaactcaaagtagttggatttaggattattttaacaagaacatccatttttcatgagcttaatattaaacccttttttgttttatatgtttttcattggaatcatgctaagatagatcatcgtaaaatagaggagaagttgaatcatgaactagcatttagaaaggaaataagaagatacatctagctagcacttagggatactgtcacaccaagatttaaggataaattcagatgcacctcatatgtgcgccatgatcaagtttcacacatataaatactcaatgtatagtagccaatgtcacaagctttattatataacgaaaatgacttacaaaataactggaataaaataaatcgaactaacataactatctccatgatgtgaatgttgtgcgcctcatcgggttttgctccgatgaaaataggagggcactcatatatgagttcatgccccgtggatccctcgataggtacatcttctcgtcgaagaagagtttctgatgggacaaactcaatgagatcgctctgggcattgctagaggtctcaactacctgcgtcacgggtgtgacatgcagattgtacacttcgacatcaagccacacaacatccttcttgacaacaactttgttccaaaagttgctgattttgggctcaccaaactgttcccaagagatgacagtttcgtgccactaagcgctgtgcagggaacgataggctatataactacatagatggtatctccaagctttggtgtcatctctagcaaattcgatgtgtatagctttgggatgctactattggagatggcgggcgatcaaaggaacgcagatcctcaagcaggaagctccagccaagcatactacccatccttcgtgtacagccagctgagccaaggagatgttgatgggatcagtgaaggtgttgatatgcatgagttagagaagaagttatgtatcatcgggctttggtgcatctagatgaagccgcaagatagaccgacgatgagcgaggtcatagagatgcttgaagctagtgttgatggcatccaaatgcctccaaggccattcttttgtgatgacgagggtgacagttcttactctacaatctctgaactgtatacaatataagagtagtagtaataaactgcacttgtgtttatgcgctcttaatgtatcatagtattaagattgcaaggttgtcataagctaaggtactagttgtatacttttattgagagtgtgataatgtgatgtcatctaaaatacctccatgaaccttttttatcaaaaactcatcaagaacattttgtttgcctacaaacagaggaccacggtcccagacattgtattcttgaaaaggaatgcactagttttatgcacgtcaatccgaaggctattacacaactttcttagttatgcataataaattaggtaaactagcatattactcgtgctaacactatgataatcaaaagaatatattctttacagctagtccaagaaacacaggagattgtatttggggatagtggcaagagtaaggtggttggtattggtaacattcctatctcaacaaagcagtcactatccaatgttctattagttgattccttaagttataaccttttgtctgttttacaactttgtgcaatgggttttgattgtctttttacaaatgtcagtgtgaaaattcttagaagggagcattcctatgttgcctttataggatagctgacaggcaagctctaccttgttgattttcaaacaagtagagtgtcatctgatacttatttagtggcaaagtccaacaagggttggctctagcattgctggctagctcatgttggcatgagaaacttgggcaaacttctaaagaatgatcacattattggactaacaaatgttatatttgagaacgacagggtttgtggagcttgccaagcaagaaaacaacatggagctctccaccatccaaagaatgcggtcaccaccaaaaaggcctttggagcttctacacatggtccttttcggaccggtggcctacctcagtattggtggtaacaaatatggcttggttattgttcatgatttttcttgcttcacttgtgtttttttctttttgggtgacaaaggtgaaacccaatagattctaaagaaggtcatgaggcgagcacaaaatgaatttgagctaaag

SEQ ID NO: 5 - DNA, 2115 bp, Zea mays L.
  source 1..2115: mol_type = genomic DNA
atgccgatgtctgattcttccttgagagctttaacggtgttatttgtgttggcagctcttgtttcagatgtagaggggcgacatcatcgtcatgtctgtcctcatttctcctgcggtggtttaagcaatatatggtatccatttcgtcggcaaggtgatccatcggggtgcggtgtccaatcgtatgagctggtttgcacggatacagatgctacaattcgcatcggcagtggaacatacaaggtgcttagcatcaactccacatattctcacttctgggttgttgatgccaacctggccgacatccagagcagttgcccccttccccggtgggattaccatgctaggaggaccagtagtaacttacatcgttggaggattgagttcagccatgatttttctaattctaatccgatgtgggtttacgctgacgcttactttgattggacccgtggcaactcatatcgtcagaggattgagttcagccatgatacccagttttcttctttggattattcggggtgggctatctttgtgaattgttctcagccaatagagaacatatataataatatatatgatgacgttgtgtatgggccggtctcttgcttgagcaattcttcttttatctacttgttaactcactggtatgctggccatgttcctgctgggagtctggagccttcatgcggttacctagccatgactcctttgggtggtccaggcatgccggtgccctcgaatataggctatccagatgttgttaagttcatgaggagtggatttgcccttcgatttcccttttcgtatggtgataacatcagagaatgtctcgcagagaatatgcgtgctttccatgaagaaccaagaaatagtacaggcatcagggaacagatcttggacattcttacattcgagacattatggttttgtgttattgatcaacttggatcaagtaataatgttgtcaaatctgttctcatcaacatcatcgtcagaataataccatttgttctgtggggtctaaaatctgcacatgttatttgcaggttcgtattgatgccgctggcagtatttgtcttcctagcctataaatactggaaaacacggataacaatagatgcagtcgagaagttcctgcgaatgcagcatatgctcgttccgatgagatatgcatacacaaacatcattgcaatcaccagccatttcagagacaagctcggacaaggaggctacggtactgtatacaagggggtgctacagccaggtgaagttcatgttgctattaagatgctaggcaactccaactgtaacggagacgagttcatcagtgaggtggccaccattggaaagatccaccatgtcaatgttgtgcgcctcattgggttttgctccgaggaaaatatcagggcacttatctatgagttcatgccccgtggatctctcgataagtacatcttctcgtcggagaagacattctcatgggacaaactcaacgagatcgctctgggcattgctagaggtctcaactacctacatcacgggtgtgatatgcagattgtacacttcgacatcaagccacacaacatccttcttgacagcaactttgttccaaaagttgctgattttgggcttgccaaactgttcccaagaggcgacactttcgtgccactgagcgctatgcggggaacgataggatatatagctccagagatggtatctcgaagctttggtgtcatctctagcaaatctgatgtgtatagctttggaatgctactgttggagatgacgggcgggcgaaggaatgcagatcctcatgcaggaagctccagtcaagcatactacccatccttggtgtacagccaactaagccaaggagatgtgggcgggatcagtaaaggtgttgatatgcacgagttagagaagaagctatgtatcattggactttggtgcatccagatgaagacgcaagatcgacagacgatgagtgaggtcatagagatgcttgaagctagtgtcgatggcatccaaatgcctccaaggccattcttttgtgatgacgagggtgatagttcttactctgcaatctctgaattggatacaatagaagagtag

SEQ ID NO: 6 - DNA, 1986 bp, synthetic construct
  misc_feature 1..1986: note = Synthetic construct
  source 1..1986: mol_type = other DNA
atggcgacgatgtctgcagcgtctcatcgctgctgtgcttcttccttgagagctttaacggtgttatttgtgttggcagctcttgtttcagatgttggcgggcgacatcatcatcatgtttgtcctccttatttctcctgcggtggttttagcaatatatcgtatccattccgtcggcaaggtgatccatcggggtgcggtgtccaatcgtatgagctggtttgcacggatacagacgctaccattcgcatcggcagtggaacgtataccgtgcttagcatcaactccacctattcttacttctgggtcgttgatgccgacctggacatccagagcagttgcccccttccctggtgggatcaccatggtgagaccagtactgccaactcatatcgtaggaggactgagttcaggccttatttcctttatccgaattcgatgtcgattatctttgtgaattgctcgaagccaatagagaacaatgatatatatgagccggtgccttgcttgagcaattcttctttcatctacttgctaactcactactcgtatggctatgctcttgctgagattctggagccctcatgcggttacctagccatgatttatttgggtggtccaggcataccggtgcccaagaatacaagctatccagatgttgttaagttaatgaggaatggatttggccttagatttccttcttcgattggtgaccgcggcatcagagaatgtttcgcagagtctgtgcgtaatttccttaaagagccaagaaagtatcagattgtggacattctaatggtcgaggaattatggtcttgttttctcgatcaacatggatcaactaataatgttgtcacttctgttatcatcgacattatcaaaacaataccaatatgtatgtggcttctgaaatctacacatgttttttgcaggcttgtattgatgccgctagcagtatttgtcttcctagccaaaacacggataacaatagatgcagtcgagaagttcctgcgaatgcagcatatgctcgttccgatgagatatgcatacacaaacatcattgcaatcaccagccatttcagagacaagctcggacaaggaggctacggtactgtatacaagggggtgctacagccaggtgaagttcatgttgctattaagatgctaggcaactccaactgtaacggagacgagttcatcagtgaggtggccaccattggaaagatccaccatgtcaatgttgtgcgcctcattgggttttgctccgaggaaaatatcagggcacttatctatgagttcatgccccgtggatctctcgataagtacatcttctcgtcggagaagacattctcatgggacaaactcaacgagatcgctctgggcattgctagaggtctcaactacctacatcacgggtgtgatatgcagattgtacacttcgacatcaagccacacaacatccttcttgacagcaactttgttccaaaagttgctgattttgggcttgccaaactgttcccaagaggcgacactttcgtgccactgagcgctatgcggggaacgataggatatatagctccagagatggtatctcgaagctttggtgtcatctctagcaaatctgatgtgtatagctttggaatgctactgttggagatgacgggcgggcgaaggaatgcagatcctcatgcaggaagctccagtcaagcatactacccatccttggtgtacagccaactaagccaaggagatgtgggcgggatcagtaaaggtgttgatatgcacgagttagagaagaagctatgtatcattggactttggtgcatccagatgaagacgcaagatcgacagacgatgagtgaggtcatagagatgcttgaagctagtgtcgatggcatccaaatgcctccaaggccattcttttgtgatgacgagggtgatagttcttactctgcaatctctgaattggatacaatagaagagtag

SEQ ID NO: 7 - protein, 661 aa, synthetic construct
  REGION 1..661: note = Synthetic construct
MATMSAASHRCCASSLRALTVLFVLAALVSDVGGRHHHHVCPPYFSCGGFSNISYPFRRQGDPSGCGVQSYELVCTDTDATIRIGSGTYTVLSINSTYSYFWVVDADLDIQSSCPLPWWDHHGETSTANSYRRRTEFRPYFLYPNSMSIIFVNCSKPIENNDIYEPVPCLSNSSFIYLLTHYSYGYALAEILEPSCGYLAMIYLGGPGIPVPKNTSYPDVVKLMRNGFGLRFPSSIGDRGIRECFAESVRNFLKEPRKYQIVDILMVEELWSCFLDQHGSTNNVVTSVIIDIIKTIPICMWLLKSTHVFCRLVLMPLAVFVFLAKTRITIDAVEKFLRMQHMLVPMRYAYTNIIAITSHFRDKLGQGGYGTVYKGVLQPGEVHVAIKMLGNSNCNGDEFISEVATIGKIHHVNVVRLIGFCSEENIRALIYEFMPRGSLDKYIFSSEKTFSWDKLNEIALGIARGLNYLHHGCDMQIVHFDIKPHNILLDSNFVPKVADFGLAKLFPRGDTFVPLSAMRGTIGYIAPEMVSRSFGVISSKSDVYSFGMLLLEMTGGRRNADPHAGSSSQAYYPSLVYSQLSQGDVGGISKGVDMHELEKKLCIIGLWCIQMKTQDRQTMSEVIEMLEASVDGIQMPPRPFFCDDEGDSSYSAISELDTIEE

SEQ ID NO: 8 - protein, 4 aa, synthetic construct
  REGION 1..4: note = peptide tag
RRRR

SEQ ID NO: 9 - protein, 6 aa, synthetic construct
  REGION 1..6: note = peptide tag
HHHHHH

SEQ ID NO: 10 - protein, 8 aa, synthetic construct
  REGION 1..8: note = peptide tag
DYKDDDDK

SEQ ID NO: 11 - protein, 8 aa, synthetic construct
  REGION 1..8: note = peptide tag
WSHPQFEK

SEQ ID NO: 12 - protein, 10 aa, synthetic construct
  REGION 1..10: note = peptide tag
EQKLISEEDL

SEQ ID NO: 13 - protein, 9 aa, synthetic construct
  REGION 1..9: note = peptide tag
YPYDVPDYA

SEQ ID NO: 14 - DNA, 22 bp, synthetic construct
  misc_feature 1..22: note = PCR primer
  source 1..22: mol_type = other DNA
cgaggaggtttcccgatattac

SEQ ID NO: 15 - DNA, 22 bp, synthetic construct
  misc_feature 1..22: note = PCR primer
  source 1..22: mol_type = other DNA
cacgtcaatccgaaggctatta

SEQ ID NO: 16 - DNA, 22 bp, synthetic construct
  misc_feature 1..22: note = PCT primer
  source 1..22: mol_type = other DNA
ttttagccctgccttcatacgc

SEQ ID NO: 17 - DNA, 23 bp, synthetic construct
  misc_feature 1..23: note = PCR primer
  source 1..23: mol_type = other DNA
cgacatcgaattcggataaagga

SEQ ID NO: 18 - DNA, 20 bp, synthetic construct
  misc_feature 1..20: note = PCR primer
  source 1..20: mol_type = other DNA
ggtggacggcgaggtcgccg

SEQ ID NO: 19 - DNA, 20 bp, synthetic construct
  misc_feature 1..20: note = PCR primer
  source 1..20: mol_type = other DNA
tcggtgacgggcaggaccgg

(19) United States
(12) Patent Application Publication
(10) Pub. No.: US 2025/0059555 A1
(43) Pub. Date: Feb. 20, 2025

(54) ZMWAK-RLK PROTEIN RELATED TO GRAY LEAF SPOT RESISTANCE, AND ENCODING GENE AND APPLICATION THEREOF

(71) Applicant: CHINA AGRICULTURAL UNIVERSITY, Beijing (CN)

(72) Inventors: Mingliang XU, Beijing (CN); Tao Zhong, Beijing (CN); Xingming Fan, Beijing (CN); Mang Zhu, Beijing (CN); Yan Zhang, Beijing (CN); Ling Xu, Beijing (CN); Li Liu, Beijing (CN)

(21) Appl. No.: 18/797,940

(22) Filed: Aug. 8, 2024

Related U.S. Application Data
(63) Continuation of application No. 17/433,159, filed on Aug. 23, 2021, now Pat. No. 12,077,769.

(30) Foreign Application Priority Data
Feb. 26, 2019 (CN) 201910140479.1

(51) Int. Cl. C12N 15/82

(52) U.S. Cl. CPC C12N 15/8282

(57) ABSTRACT

The present invention discloses a ZmWAK-RLK protein related to gray leaf spot resistance, and an encoding gene and application thereof. The present invention sets forth a protein obtained from a maize inbred line Y32, named ZmWAK-RLK, and being the protein shown in sequence 1 of the sequence listing. A nucleic acid molecule for encoding the ZmWAK-RLK protein is likewise considered to be within the scope of protection of the present invention. The present invention likewise sets forth a method for preparing a transgenic plant, comprising the following step: introducing said nucleic acid molecule into a starting plant, thus obtaining a transgenic plant having enhanced resistance to gray leaf spot. The present invention additionally sets forth a plant breeding method, comprising the following step: increasing the content and/or activity of the ZmWAK-RLK protein in a plant of interest, thus increasing the gray leaf spot resistance of the plant of interest. The present invention has great value when applied to the breeding of maize resistant to gray leaf spot.

### CROSS-REFERENCE TO RELATED APPLICATIONS

This application is a continuation of U.S. application Ser. No. 17/433,159, filed on Aug. 23, 2021, now U.S. Pat. No. 12,077,769, which is a national stage entry under 35 USC § 371 of PCT International Application Number PCT/CN2020/076320, filed Feb. 24, 2020, which claims priority to Chinese Patent Application Number 201910140479.1 filed Feb. 26, 2019, the entire disclosures of which are incorporated herein by reference.

### INCORPORATION BY REFERENCES OF MATERIAL SUBMITTED ELECTRONICALLY

Incorporated by reference in its entirety is a computer-readable nucleotide/amino acid sequence listing submitted as a 39 kilobytes xml document named “416902.xml”, and created on Nov. 10, 2024.

### TECHNICAL FIELD

The present invention belongs to the field of biotechnology, and specifically relates to a ZmWAK-RLK protein related to gray leaf spot resistance, and an encoding gene and an application thereof.

### BACKGROUND ART

Maize gray leaf spot (GLS) is a maize leaf disease that affects the yield and quality of maize. In the 1920s, gray leaf spot was first discovered in Alexandria County, Illinois, USA. Later on, it gradually developed into a serious global plant leaf disease. Gray leaf spot is widely distributed in the main maize producing regions in the United States, Asia, Europe, and Africa. When the disease occurs, gray leaf spot can cause a 20-60% reduction in production. In the case of a severe disease, the reduction can reach 100%, causing serious economic losses to maize production.

Maize gray leaf spot is a fungal disease, and it is generally believed that the pathogenic fungi mainly include Cercospora zeae-maydis (Czm) and Cercospora zeina (Cz). In 2013, Liu et al. collected extensive disease samples from areas in China where maize gray leaf spot occurred, and obtained a large number of strains through the method of monospore isolation. By means of the methods of pathogen morphology, cultivation characteristic growth status, and molecular biology, they accurately identified the pathogenic species of maize gray leaf spot in different regions in China. Cercospora zeae-maydis is the cause of maize gray leaf spot disease in northern China, while Cercospora zeina is the cause of maize gray leaf spot disease in southwestern China.

According to reports, the resistance of maize to gray leaf spot belongs to quantitative inheritance, which is controlled by multiple genes with additive effects. In this regard, if the gray leaf spot resistance gene can be cloned and introduced into existing inbred lines using a molecular marker-assisted selection technology, this will be able to improve the gray leaf spot resistance of those popularized varieties.

### SUMMARY OF THE INVENTION

The present invention provides a gray leaf spot resistance-related protein ZmWAK-RLK, and an encoding gene and an application thereof.

The protein provided by the present invention is obtained from the maize inbred line Y32 and named as ZmWAK-RLK protein, which is as follows: (a1), or (a2), or (a3), or (a4), or (a5):
(a1) a protein represented by SEQ ID NO: 1 in the sequence listing;
(a2) a protein represented by SEQ ID NO: 7 in the sequence listing;
(a3) a fusion protein obtained by attaching a tag to an N-terminus or/and a C-terminus of the protein of (a1) or (a2);
(a4) a protein related to plant gray leaf spot resistance obtained by substituting and/or deleting and/or adding one or a plurality of amino acid residues to the protein of (a1) or (a2); and
(a5) a protein related to plant gray leaf spot resistance obtained from maize and having a homology of more than 90%, 91%, 92%, 93%, 94%, 95%, 96%, 97%, 98%, or 99% with the protein of (a1) or (a2).

The tags are as shown in Table 1 below.

TABLE 1
Tag sequences

| Tag | Residues | Sequence |
|---|---|---|
| Poly-Arg | 5 to 6 (typically 5) | RRRR (SEQ ID NO: 8) |
| Poly-His | 2 to 10 (typically 6) | HHHHHH (SEQ ID NO: 9) |
| FLAG | 8 | DYKDDDDK (SEQ ID NO: 10) |
| Strep-tag II | 8 | WSHPQFEK (SEQ ID NO: 11) |
| c-myc | 10 | EQKLISEEDL (SEQ ID NO: 12) |
| HA | 9 | YPYDVPDYA (SEQ ID NO: 13) |

The protein can be synthesized artificially, or its encoding gene can be synthesized first, and then the protein can be obtained by biological expression.

The nucleic acid molecule encoding the ZmWAK-RLK protein also falls within the scope of protection of the present invention.

The nucleic acid molecule is (b1), or (b2), or (b3), or (b4), or (b5), or (b6);

(b1) a DNA molecule with an encoding region that is represented by nucleotides 87 to 2084 in SEQ ID NO: 2 in the sequence listing;

(b2) a DNA molecule represented by SEQ ID NO: 2 in the sequence listing;
(b3) a DNA molecule represented by SEQ ID NO: 3 in the sequence listing;
(b4) a DNA molecule whose encoding region is represented by SEQ ID NO: 6 in the sequence listing;

(b5) a DNA molecule that is derived from maize, has a homology of more than 90%, 91%, 92%, 93%, 94%, 95%, 96%, 97%, 98%, or 99% with (b1), or (b2), or (b3), or (b4) and encodes the protein; and
(b6) a DNA molecule that hybridizes to a nucleotide sequence defined by (b1), or (b2), or (b3), or (b4) under a stringent condition, and encodes the protein.

The stringent condition mentioned above is as follows: in a solution of 2×SSC, 0.1% SDS, hybridizing is performed at 68° C. the membrane is washed twice for 5 min each time, then hybridizing is performed again in a solution of 0.5×SSC, 0.1% SDS at 68° C., and the membrane is then washed twice for 15 min each time.

A DNA molecule, expression cassette, recombinant vector, or recombinant microorganism containing the nucleic acid molecule all falls within the scope of protection of the present invention.

The DNA molecule containing the nucleic acid molecule may specifically be as shown in Sequence 4 in the sequence listing.

Existing expression vectors can be used to construct a recombinant expression vector containing the nucleic acid molecule. When using the nucleic acid molecule to construct a recombinant expression vector, any enhanced, constitutive, tissue-specific, or inducible promoter can be added before its transcription initiation nucleotide, and they can be used alone or in combination with other plant promoters. In addition, when using the nucleic acid molecule to construct a recombinant expression vector, enhancers, including translation enhancers or transcription enhancers, can also be used. These enhancer regions can be ATG start codons or adjacent region start codons, but they must be in the same reading frame as the coding sequence in order to ensure correct translation of the entire sequence. The sources of the translation control signals and start codons are extensive, and they can be natural or synthetic. The translation initiation region can be derived from a transcription initiation region or a structural gene. In order to facilitate the identification and screening of transgenic plants or transgenic microorganisms, the expression vectors used can be processed. For example, genes expressing enzymes or luminescent compounds that can produce color changes in plants or microorganisms, resistant antibiotic markers, or chemical reagent resistant marker genes can be added herein. Considering the safety of the transgenes, it is possible to directly screen transformed plants or microorganisms by phenotype without adding any selectable marker genes.

The recombinant expression vector may specifically be a recombinant plasmid obtained by inserting the double-stranded DNA molecule shown in Sequence 4 in the sequence listing into the multiple cloning site (for example, the BamHI site) of the pCAMBIA3301 vector.

The recombinant expression vector may specifically be a recombinant plasmid obtained by inserting the double-stranded DNA molecule shown in nucleotides 87 to 2084 of Sequence 2 in the sequence listing into the multiple cloning site (for example, the Xcm I restriction site) of the pBCXUN vector.

The recombinant expression vector may specifically be a recombinant plasmid obtained by inserting the recombinant double-stranded DNA molecule shown in Sequence 6 in the sequence listing into the multiple cloning site (for example, the Xcm I restriction site) of the pBCXUN vector.

The present invention further sets forth an application of the ZmWAK-RLK protein, which is the following (c1), or (c2), or (c3), or (c4):
(c1) to regulate the resistance of a plant to gray leaf spot;
(c2) to increase the resistance of a plant to gray leaf spot;
(c3) to regulate the resistance of a plant to a disease caused by Cercospora zeina;
(c4) to increase the resistance of a plant to a disease caused by Cercospora zeina.

The present invention further sets forth an application of the nucleic acid molecule or the DNA molecule containing the nucleic acid molecule, which is the following (d1), or (d2), or (d3), or (d4):

(d1) to cultivate a transgenic plant with altered resistance to gray leaf spot;
(d2) to cultivate a transgenic plant with increased resistance to gray leaf spot;
(d3) to cultivate a transgenic plant with altered resistance to a disease caused by Cercospora zeina;
(d4) to cultivate a transgenic plant with increased resistance to a disease caused by Cercospora zeina.

The application of the nucleic acid molecule also includes an implementation method of using the gene through the CRISPS/CAS9 technology, for example, genome fragment reset (to introduce disease-resistant alleles into the susceptible genome), allele exchange (to replace susceptible alleles with disease-resistant alleles), changing susceptible alleles into disease-resistant alleles through gene editing, and so on.

The application of the nucleic acid molecule also includes other implementation methods aimed at enhancing the expression of the nucleic acid molecule. For example, the expression of the nucleic acid molecule can be increased by promoter replacement, the expression of the nucleic acid molecule can be increased by introducing an enhancer, and so on.

The present invention also sets forth a method for preparing a transgenic plant, which includes the following step: introducing the nucleic acid molecule or the DNA molecule containing the nucleic acid molecule into a starting plant to obtain a transgenic plant with increased resistant to gray leaf spot. The nucleic acid molecule can be specifically introduced into the starting plant through any one of the above-mentioned recombinant expression vectors. The recombinant expression vector carrying the nucleic acid molecule can be transformed into the starting plant by conventional biological methods such as Ti plasmid. Ri plasmid, plant virus vector, direct DNA transformation, microinjection, electrical conduction, and agrobacterium mediation. By crossing the transgenic plants with an existing maize variety (including single crosses and multiple crosses, such as three consecutive crosses), the obtained transgenic progeny plants are also transgenic plants with increased resistance to gray leaf spot. The existing maize variety may specifically be a maize inbred line Q11.

The present invention also sets forth a plant breeding method, which includes the following step: increasing the content and/or activity of the ZmWAK-RLK protein in a target plant, thereby increasing the resistance of the target plant to gray leaf spot.

The present invention also sets forth a method for preparing a transgenic plant, which includes the following step: introducing the nucleic acid molecule or the DNA molecule containing the nucleic acid molecule into a starting plant to obtain a transgenic plant with increased disease resistance; the disease resistance is the disease resistance to a disease caused by Cercospora zeina. The nucleic acid molecule can be specifically introduced into the starting plant through any one of the above-mentioned recombinant expression vectors. The recombinant expression vector carrying the nucleic acid molecule can be transformed into the starting plant by conventional biological methods such as Ti plasmid, Ri plasmid, plant virus vector, direct DNA transformation, microinjection, electrical conduction, and agrobacterium mediation. By crossing the transgenic plants with an existing maize variety (including single crosses and multiple crosses, such as three consecutive crosses), the obtained transgenic progeny plants are also transgenic plants with increased resistance to gray leaf spot. The existing maize variety may specifically be a maize inbred line Q11.

The present invention also sets forth a plant breeding method, which includes the following step: increasing the content and/or activity of the ZmWAK-RLK protein in a target plant, thereby increasing the disease resistance of the target plant; the disease resistance is the disease resistance to a disease caused by Cercospora zeina.

Any of the above-mentioned plants is a dicotyledonous plant or a monocotyledonous plant. The monocotyledonous plant may be a gramineous plant. The gramineous plant may be a plant of the genus Zea. The Zea plant may specifically be maize, such as maize inbred line B73 or maize inbred line B73-329.

Any of the above gray leaf spots may specifically be gray leaf spot caused by Cercospora zeina.

### BRIEF DESCRIPTION OF THE DRAWINGS AND LEGENDS

FIG. 1 is the PCR identification results of some plants in Example 2. The arrow marks the target band (1197 bp). The leftmost lane is the molecular weight standard (M), each of the remaining lanes corresponds to a plant (numbered 1 to 18). If the 1197 bp amplification product is obtained, and the PCR identification is positive, the plant is a transgenic plant. If no amplification product is obtained, and the PCR identification is negative, the plant is a non-transgenic plant.

FIG. 2 is a photograph of representative leaves of each level: the disease-resistant parent Y32 has fewer disease spots on the leaves, while the susceptible parent Q11 has more disease spots on the leaves.

FIG. 3 is the results of the identification of disease resistance of the offspring separated from backcrossing in Example 2. In the BC₁F₁ generation and the BC₃F₁ generation, the disease index of the non-transgenic plants and that of the transgenic plants in the offspring of the two transgenic events WAK1-15 and WAK1-17 were counted respectively. The gray bars are non-transgenic plants, and the black bars are transgenic plants. The numbers in the bar graph indicate the numbers of plants. *: P<0.05; **: P<0.01.

FIG. 4 is the results of the identification of disease resistance of the homozygous transgenic lines in Example 2. For the disease index of a homozygous transgenic positive plant and that of a transgenic receptor material (B73-329), it can be seen that the disease index of the homozygous transgene positive plant is significantly lower than that of the transgenic receptor material. The gray bar is B73-329, which is a transgenic receptor material. The black bar is a homozygous line of the transgenic plants. The numbers in the bar graph indicate the numbers of plants.

FIG. 5 is the PCR identification results of some plants in Example 3. The arrow marks the target band (530 bp). The leftmost lane is the molecular weight standard (M). Each of the remaining lanes corresponds to a plant (numbered 1 to 19). If the 530 bp amplification product is obtained, and the PCR identification is positive, the plant is a transgenic plant. If no amplification product is obtained, and the PCR identification is negative, the plant is a non-transgenic plant.

FIG. 6 is the results of the identification of disease resistance of the offspring separated from backcrossing in Example 3. In the BC₁F₁ generation and the BC₃F₁ generation, the disease index of the non-transgenic plants and that of the transgenic plants in the offspring of the three transgenic events WAK2-6, WAK2-7, and WAK2-8. The gray bars are non-transgenic plants; the black bars are transgenic plants. The numbers in the bar graph indicate the numbers of plants, *: P<0.05; ***: P<0.001; NS: No significant difference.

FIG. 7 is the disease resistance identification results (disease index) of the B73 background complementary transgenic homozygous lines in Example 4. The gray bar is B73, which is the transgenic receptor material. The black bars are the homozygous transgenic lines C #1, C #2, and C #3. The numbers in the bar graph indicate the numbers of plants. *: P<0.05; **: P<0.01.

FIG. 8 is the disease resistance identification results (disease index) of the B73 background overexpression transgenic homozygous lines in Example 5. The gray bar is B73, which is the receptor material. The black bars are the homozygous transgenic lines O #1, O #2, O #3, and O #4. The numbers in the bar graph indicate the numbers of plants. *: P<0.05.

FIG. 9 is the PCR identification results of some plants in Example 6. The arrow marks the target band (357 bp). The leftmost lane is the molecular weight standard (M). Each of the remaining lanes corresponds to a plant (numbered 1 to 14). If the 357 bp amplification product is obtained, and the PCR identification is positive, the plant is a transgenic plant. If no amplification product is obtained, and the PCR identification is negative, the plant is a non-transgenic plant.

FIG. 10 is the disease resistance identification results (disease index) of the Q11 background chimeric gene overexpression plants in Example 6. In the BC₂F₁ generation, the disease index of the non-transgenic plants and that of the transgenic plants in the offspring of the three transgenic events of R1, R2, and R3 were counted. The gray bars are non-transgenic plants, and the black bars are transgenic plants. The numbers in the bar graph indicate the numbers of plants. *: P<0.05; **: P<0.01.

### DESCRIPTION OF THE PREFERRED EMBODIMENTS

The following examples facilitate a better understanding of the present invention, but do not limit the present invention. The experimental methods in the following examples, unless otherwise specified, are all conventional methods. The experimental materials used in the following examples, unless otherwise specified, are all purchased from conventional biochemical reagent stores. The quantitative experiments in the following examples are all set to repeat the experiments three times, and the results are averaged.

The maize inbred line Y32 is a maize inbred line with high resistance to gray leaf spot of maize. The maize inbred line Y32 (line Y32) is described in the following literature: Theoretical and Applied Genetics, 2012, 25 (8): 1797-1808. Zhang, Y., et al. “QTL mapping of resistance to gray leaf spot in maize.”

The maize inbred line Q11 is a maize inbred line highly susceptible to gray leaf spot. The maize inbred line Q11 (line Q11) is described in the following literature: Theoretical and Applied Genetics, 2012, 25 (8): 1797-1808. Zhang. Y., et al. “QTL mapping of resistance to gray leaf spot in maize.”

The maize inbred line B73-329 (B73-329 inbred lines) is described in the following literature: New Phytologist, 2018, 217 (3): 1161-1176. Zhang, M., et al. “A retrotransposon in an HKT1 family sodium transporter causes variation of leaf Na⁺ exclusion and salt tolerance in maize.”

The maize inbred line B73 (B73 inbred lines) is described in the following literature: Plant Science, 1985, 41 (2): 140. Everett. N. P., et al. “Biochemical markers of embryogenesis in tissue cultures of the maize inbred B73.”

Cercospora zeina is described in the following literature: Plant Disease, 2013, 97 (12): 1656-1656. Liu, K. J., et al. “First Report of Gray Leaf Spot of Maize Caused by Cercospora zeina in China.”

The pCAMBIA3301 vector (bivalent expression vector pCAMBIA3301) is described in the following literature: Theoretical and Applied Genetics 131.10 (2018): 2145-2156. Zhu, X. et al. “Pyramiding of nine transgenes in maize generates high-level resistance against necrotrophic maize pathogens.”

The pBCXUN vector is described in the following literature: Journal of integrative plant biology 61.6 (2019): 691-705. Qin. Y. J., et al. “ZmHAK5 and ZmHAK1 function in K⁺ uptake and distribution in maize under low K⁺ conditions.”

### Example 1 Discovery of ZmWAK-RLK Protein and an Encoding Gene Thereof

The maize inbred line Y32 with high resistance to gray leaf spot (as the donor parent) and the maize inbred line Q11 that is highly susceptible to gray leaf spot (as the recurrent parent) were used to construct the initial positioning population and the fine positioning population. In addition, qRgls1 was mapped between IDP2 and M2 of maize chromosome 8. The physical location is about 120 kb.

A finely positioned molecular marker located in the QTL-qRgls1 region was used, and the Y32 BAC library of the disease-resistant parents was screened by PCR. The BAC clone fingerprint analysis was used to construct BAC contigs covering the entire gene portion. The clones that could cover the least gene region were selected for sequencing. Through sequence alignment and expression analysis, a new gene was found, which encodes the protein shown in Sequence 1 in the sequence listing.

The protein shown in Sequence 1 in the sequence listing was named ZmWAK-RLK protein. The gene encoding the ZmWAK-RLK protein was named ZmWAK-RLK gene. The ZmWAK-RLK gene in the cDNA of the maize inbred line Y32 was shown in Sequence 2 in the sequence listing (where the nucleotides of 87 to 2084 are the open reading frame) (in Sequence 2, the nucleotides of 87 to 1058 are used for the construction of the chimeric gene). The ZmWAK-RLK gene in the genomic DNA of the maize inbred line Y32 was shown in Sequence 3 in the sequence listing. The open reading frame sequence of the ZmWAK-RLK gene in the cDNA of the maize inbred line Q11 was shown in Sequence 5 in the sequence listing (in Sequence 5, the nucleotides of 1102 to 2115 were used to construct the chimeric gene). The chimeric gene was shown in Sequence 6 in the sequence listing, and expressed the chimeric protein shown in Sequence 7 in the sequence listing.

### Example 2 Verifying the Function of a 7.2 kb Fragment

### I. Obtaining Transgenic Plants

1. A fragment of about 7.2 kb in the maize inbred line Y32 (the fragment is shown in Sequence 4 in the sequence listing. In Sequence 4, the nucleotides 1 to 2103 were the promoter, and the nucleotides 2104 to 4316 were the same as Sequence 3 in the sequence listing) was inserted into the BamH I restriction site of the pCAMBIA3301 vector to obtain a recombinant plasmid.

2. The recombinant plasmid obtained in step 1 was introduced into Agrobacterium EHA105 to obtain a recombinant Agrobacterium.

3. The recombinant Agrobacterium obtained in step 2 was taken, and the Agrobacterium mediated method was used to genetically transform the immature embryos of the maize inbred line B73-329 to obtain TO generation plants.

4. The TO generation plants were selfed, the seeds were harvested, and the seeds were cultivated into plants, that is, the T1 generation plants.

5. The T1 generation plants were identified by PCR, and transgenic plants were screened. The transgenic plants selected from the T1 generation plants were the T1 transgenic plants. A number of transgenic plants were selected from the T1 generation plants, two of which were named WAKI-15 plant and WAKI-17 plant.

PCR identification method: plant leaves were taken, genomic DNA was extracted therefrom, and a primer pair composed of F1 and RI was used for PCR amplification. If the 1197 bp amplification product was obtained, and the PCR identification was positive, the plant was a transgenic plant. If no amplification product was obtained, and the PCR identification was negative, the plant was a non-transgenic plant.

F1: CGAGGAGGTTTCCCGATATTAC (SEQ ID NO: 14):
R1: CACGTCAATCCGAAGGCTATTA (SEQ ID NO: 15).

FIG. 1 is the PCR identification results of some plants. The arrow marks the target band. The leftmost lane (M) is the molecular weight standard, and each of the remaining lanes (numbered 1 to 18) corresponds to a plant.

### II. Obtaining B73-329 Genetic Background Transgenic Homozygous Line

The T1 transgenic plants were selfed and the seeds were harvested. The seeds were cultivated into plants, that is, the T2 generation plants. The T2 generation plants were selfed and the seeds were harvested. The seeds were cultivated into plants, that is, the T3 generation plants.

The T3 generation plants were identified by PCR (the PCR identification method was the same as 5 in step I).

For a T2 generation plant, if the T3 generation plants obtained by its selfing were all transgenic plants, this T2 generation plant was a homozygous transgenic plant. The offspring obtained by selfing of the homozygous transgenic plant were homozygous transgenic lines.

III. Obtaining the Offspring Separated from Backcrossing

The PCR identification method was the same as 5 in step I.

1. The WAKI-15 plant (or WAKI-17 plant) as the male parent was crossed with the maize inbred line Q11 as the female parent. The seeds were harvested, and cultivated into plants, that is, the BC1F1 plants. The transgenic plants and non-transgenic plants were identified and screened by PCR.

2. The WAKI-15 plant (or WAKI-17 plant) as the male parent was crossed with the maize inbred line Q11 as the female parent. The seeds were harvested, and cultivated into plants, that is, the BC1F1 plants. The transgenic plants were identified and screened by PCR. The transgenic plant of the BC1F1 plants was used as the male parent to cross with the maize inbred line Q11 as the female parent. The seeds were harvested, and cultivated into plants, that is, the BC: F₁ plants. The transgenic plants were identified and screened by PCR. The transgenic plant of the BC₂F₁ plants was used as the male parent to cross with the maize inbred line Q11 as the female parent. The seeds were harvested, and cultivated into plants, that is, the BC₃F₁ plants. The transgenic plants and non-transgenic plants were identified and screened by PCR.

### IV. Identifying Plant Disease Resistance

### 1. Methods of Identification of Disease Resistance

The disease resistance identification was carried out in Shangzhuang Experimental Base of China Agricultural University.

The pathogen of gray leaf spot: Cercospora zeina.

The plants as the experimental materials were cultivated under normal conditions, the pathogen was inoculated at the 10-leaf stage, and then the plants were cultivated again under the normal conditions. The phenotypic investigation was carried out two weeks after pollination. A graded survey was used to calculate the disease index (DSI). The specific method of inoculating the pathogen (method of pathogen solution infusion) was as follow: sterile water was used to suspend the spores of the pathogen of gray leaf spot to obtain a spore suspension with a spore concentration of 1×10⁵ cfu/mL. A syringe was then used to infuse the spore suspension into the leaf heart of the maize plant, and 5 mL was used for each maize.

The grading standard of the disease levels (X represents the percentage of the diseased spot area to the leaf area):
Level 1 (assigned value was 0)): X≤5%;
Level 3 (assigned value was 0.25): 5%<X≤10%;
Level 5 (assigned value was 0.5): 10%<X≤30%;
Level 7 (assigned value was 0.75): 30%<X≤70%;
Level 9 (assigned value was 1): 70%<X≤100%.

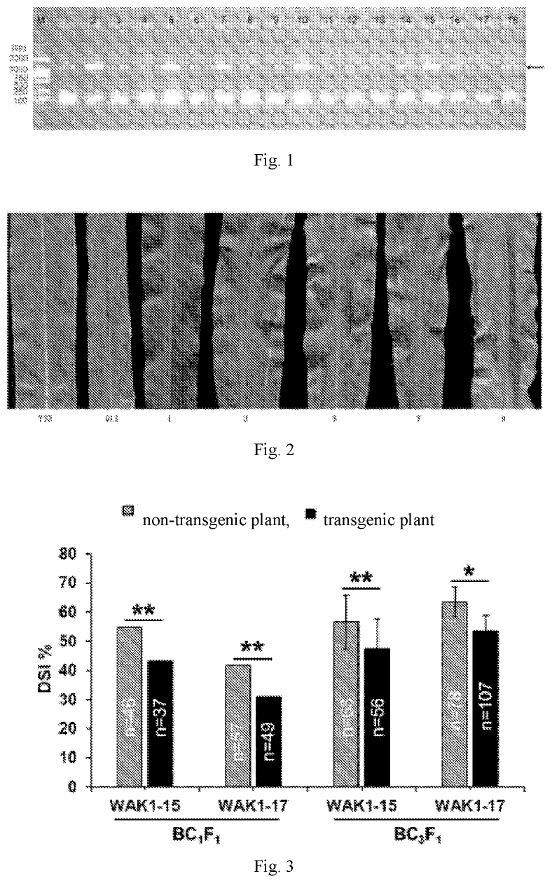

See FIG. 2 for photos of representative leaves of all levels.

Disease index (DSI) (%)=Σ(assigned value corresponding to a disease level x the number of plants at this level)×100/1×total number of plants

2. Identifying the Disease Resistance of the Offspring Separated from Backcrossing (BC₁F₁ Plants and BC₃F₁ Plants)

The first group of test materials: the transgenic plants and non-transgenic plants of the BC₁F₁ plants obtained with the WAK1-15 plant as the male parent as shown in 1 in step III, and the transgenic plants and non-transgenic plants of the BC₁F₁ plants obtained with the WAK1-17 plant as the male parent as shown in 1 in step III;

The second group of test materials: the transgenic plants and non-transgenic plants of the BC₃F₁ plants obtained with the WAK1-15 plant as the male parent as shown in 2 in step III, and the transgenic plants and non-transgenic plants of the BC₃F₁ plants obtained with the WAK1-17 plant as the male parent as shown in 2 in step III.

The first group of test materials was identified for disease resistance according to the method in step 1. The disease index of the transgenic plants was significantly lower than that of the non-transgenic plants, and DSI decreased by 10.5-11.6%.

The second group of test materials was identified for disease resistance according to the method in step 1. The disease index of the transgenic plants was significantly lower than that of the non-transgenic plants, and DSI decreased by 9.5-10.6%.

The results were shown in FIG. 3 (the gray bars are non-transgenic plants; the black bars are transgenic plants. The numbers in the bar graph indicate the numbers of plants. *: P<0.05; **: P<0.01.)

### 3. Identifying Disease Resistance of Homozygous Transgenic Lines

Test materials: T3 generation plants of the homozygous transgenic line obtained in step II and the maize inbred line B73-329 plants.

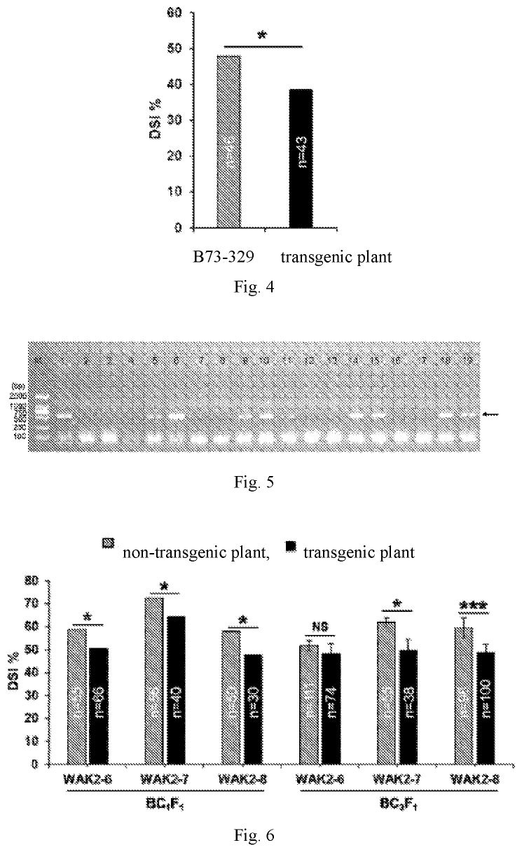

The test materials were identified for disease resistance according to the method in step 1. The results are shown in FIG. 4 (The gray bar is B73-329, which is the genetically modified receptor plant. The black bar is a homozygous line of the transgenic plants. The numbers in the bar graph indicate the numbers of plants. *: P<0.05). The disease index of transgenic plants was significantly lower than that of the maize inbred line B73-329 plants, and DSI was reduced by 9.5%.

The above results indicate that the ZmWAK-RLK gene is a functional gene of QTL-qRls1. Introducing the ZmWAK-RLK gene into maize can significantly reduce its gray leaf spot disease index by about 10%.

### Example 3 Verifying the Function of the Open Reading Frame

### I. Obtaining Transgenic Plants

1. The double-stranded DNA molecule shown as the nucleotides 87 to 2084 of Sequence 2 in the sequence listing was inserted into the Xcm I restriction site of the pBCXUN vector to obtain a recombinant plasmid.

2. The recombinant plasmid obtained in step I was introduced into Agrobacterium EHA105 to obtain a recombinant Agrobacterium.

3. The recombinant Agrobacterium obtained in step 2 was taken, and the Agrobacterium mediated method was used to genetically transform the immature embryos of the maize inbred line B73-329 to obtain TO generation plants.

4. The TO generation plants were selfed, and the seeds were harvested. The seeds were cultivated into plants, that is, the T1 generation plants.

5. The T1 generation plants were identified by PCR, and transgenic plants were screened. The transgenic plants selected from the T1 generation plants were the T1 transgenic plants. A number of transgenic plants were selected from the T1 generation plants, three of which were named WAK2-6 plant, WAK2-7 plant, and WAK2-8 plant.

PCR identification method: plant leaves were taken, genomic DNA was extracted therefrom, and a primer pair composed of F2 and R2 was used for PCR amplification. If the 530 bp amplification product was obtained, and the PCR identification was positive, the plant was a transgenic plant. If no amplification product was obtained, and the PCR identification was negative, the plant was a non-transgenic plant.

F2: TTTTAGCCCTGCCTTCATACGC (SEQ ID NO: 16):
R2: CGACATCGAATTCGGATAAAGGA (SEQ ID NO: 17).

The PCR identification results of some plants are shown in FIG. 5. The arrow marks the target band. The leftmost lane is the molecular weight standard (M). Each of the remaining lanes corresponds to a plant (numbered 1 to 19).

II. Obtaining the Offspring Separated from Backcrossing

The PCR identification method was the same as 5 in step I.

1. The WAK2-6 plant (or WAK2-7 plant or WAK2-8 plant) as the male parent was crossed with the maize inbred line Q11 as the female parent. The seeds were harvested, and cultivated into plants, that is, the BC1F1 plants. The transgenic plants and non-transgenic plants were identified and screened by PCR.

2. The WAK2-6 plant (or WAK2-7 plant or WAK2-8 plant) as the male parent was crossed with the maize inbred line Q11 as the female parent. The seeds were harvested, and cultivated into plants, that is, the BC₁F₁ plants. The transgenic plants were identified and screened by PCR. The transgenic plant of the BC₁F₁ plants was used as the male parent to cross with the maize inbred line Q11 as the female parent. The seeds were harvested, and cultivated into plants, that is, the BC₂F₁ plants. The transgenic plants were identified and screened by PCR. The transgenic plant of the BC₂Fj plants was used as the male parent to cross with the maize inbred line Q11 as the female parent. The seeds were harvested, and cultivated into plants, that is, the BC₃F₁ plants. The transgenic plants and non-transgenic plants were identified and screened by PCR.

### III. Identifying Plant Disease Resistance

### 1. Methods of Identification of Disease Resistance

The method was the same as 1 in step IV of Example 2.

2. Identifying the Disease Resistance of the Offspring Separated from Backcrossing (BC₁F₁ Plants and BC₃F₁ Plants)

The first group of test materials: the transgenic plants and non-transgenic plants of the BC₁F₁ plants obtained with the WAK2-6 plant as the male parent as shown in 1 in step II, the transgenic plants and non-transgenic plants of the BC₁F₁ plants obtained with the WAK2-7 plant as the male parent as shown in 1 in step II, and the transgenic plants and non-transgenic plants of the BC₁F₁ plants obtained with the WAK2-8 plant as the male parent as shown in 1 in step II;

The second group of test materials: the transgenic plants and non-transgenic plants of the BC₃F₁ plants obtained with the WAK2-6 plant as the male parent as shown in 2 in step II, the transgenic plants and non-transgenic plants of the BC₃F₁ plants obtained with the WAK2-7 plant as the male parent as shown in 2 in step II, and the transgenic plants and non-transgenic plants of the BC₃F₁ plants obtained with the WAK2-8 plant as the male parent as shown in 2 in step II.

The first group of test materials was identified for disease resistance according to the method in step 1. The disease index of the transgenic plants was significantly lower than that of the non-transgenic plants, and DSI decreased by 8.3-10.5%.

The second group of test materials was identified for disease resistance according to the method in step 1. The disease index of the transgenic plants was significantly lower than that of the non-transgenic plants, and DSI decreased by 10.8-11.9%.

The results were shown in FIG. 6 (the gray bars are non-transgenic plants, and the black bars are transgenic plants. The numbers in the bar graph indicate the numbers of plants. *: P<0.05; ***: P<0.001; NS: no significant difference).

The above results indicate that the ZmWAK-RLK gene is a functional gene in the main QTL-qRgls1 region for resistance to gray leaf spot. When it is backcrossed to be introduced to the Q11 genetic background, it can significantly improve the resistance of maize to gray leaf spot.

### Example 4 Verifying the Function of the 7.2 kb Fragment on the B73 Genetic Background

### I. Obtaining Transgenic Plants

1. This step was the same as 1 in step 1 in Example 2.

2. This step was the same as 2 in step I in Example 2.

3. The recombinant Agrobacterium obtained in step 2 was taken, and the Agrobacterium mediated method was used to genetically transform the immature embryos of maize inbred line B73 to obtain the T0 generation plants.

4. The T0 generation plants were selfed, the seeds were harvested, and the seeds were cultivated into plants, that is, the T1 generation plants.

5. The T1 generation plants were identified by PCR, and transgenic plants were screened.

The PCR identification method was the same as 5 in Step I of Example 2.

The transgenic plants selected from the T1 generation plants were the T1 transgenic plants.

### II. Obtaining Homozygous Transgenic Lines of B73 Genetic Background

The method was the same as step II of Example 2.

Three homozygous transgenic line materials were obtained, C #1 line. C #2 line, and C #3 line.

### III. Identifying Plant Disease Resistance

### 1. Methods of Identification of Disease Resistance

The method was the same as 1 in step IV of Example 2.

### 2. Identifying Disease Resistance of Homozygous Transgenic Lines of the B73 Genetic Background

Test materials: T3 generation plants of C #1 line, T3 generation plants of C #2 line, T3 generation plants of C #3 line, and maize inbred line B73 plants.

The test materials were identified for disease resistance according to the method in step 1.

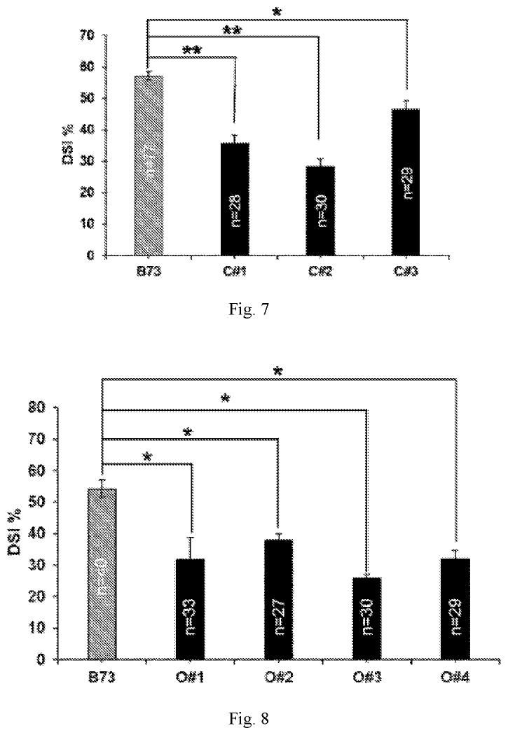

Compared with the receptor material (maize inbred line B73 plants), the plant disease index of the C #1 line was significantly reduced (the reduction was 21.3%), the plant disease index of the C #2 line was significantly reduced (the reduction was 28.6%), and the plant disease index of the #3 line was also significantly reduced (the reduction was 10.5%). The results were shown in FIG. 7 (The gray bar is the transgenic receptor material, and the black bars are the homozygous transgenic plants. The numbers in the bar graph indicate the numbers of plants. *: P<0.05; **: P<0.01).

Overall, the transfer of the disease resistance gene ZmWAK-RLK can significantly reduce the disease index of maize gray leaf spot and increase the resistance of maize to gray leaf spot.

### Example 5 Verifying the Function of the Open Reading Frame on the B73 Genetic Background

### 1. Obtaining Transgenic Plants

1. This step was the same as 1 in step I in Example 3.

2. This step was the same as 2 in step I in Example 3.

3. The recombinant Agrobacterium obtained in step 2 was taken, and the Agrobacterium mediated method was used to genetically transform the immature embryos of maize inbred line B73 to obtain the TO generation plants.

4. The TO generation plants were selfed, the seeds were harvested, and the seeds were cultivated into plants, that is, the T1 generation plants.

5. The T1 generation plants were identified by PCR, and transgenic plants were screened.

The PCR identification method was the same as 5 in Step I of Example 5.

The transgenic plants selected from the T1 generation plants were the T1 transgenic plants.

### II. Obtaining Homozygous Transgenic Lines of B73 Genetic Background

The method was the same as step II of Example 2.

Four homozygous transgenic line materials were obtained, O #1 line, O #2 line, O #3 line, and O #4 line.

### III. Identifying Plant Disease Resistance

### 1. Methods of Identification of Disease Resistance

The method was the same as 1 in step IV of Example 2.

### 2. Identifying Disease Resistance of Homozygous Transgenic Lines of the B73 Genetic Background

Test materials: T3 generation plants of O #1 line. T3 generation plants of O #2 line, T3 generation plants of O #3 line, T3 generation plants of O #4 line, and maize inbred line B73 plants.

The test materials were identified for disease resistance according to the method in step 1.

Compared with the receptor material (maize inbred line B73 plants), the plant disease index of the O #1 line was significantly reduced (the reduction was 22.5%), the plant disease index of the O #2 line was significantly reduced (the reduction rate was 16.3%), the plant disease index of the O #3 line was significantly reduced (the reduction rate was 28.3%), and the plant disease index of the O #4 line was significantly reduced (the reduction rate was 22.2%). The results were shown in FIG. 8 (The gray bar is the transgenic recipient material, and the black bars are the homozygous transgenic plants. The numbers in the bar graph indicate the numbers of plants. *: P<0.05).

The results demonstrate that the ZmWAK-RLK gene is a functional gene in the main QTL-qRgls1 region for resistance to gray leaf spot, which can significantly improve the resistance of maize to gray leaf spot.

### Example 6 Verifying the ZmWAK-RLK Functioning Section in Y32 on the Q11 Background

### I. Obtaining Transgenic Plants

1. The double-stranded DNA molecule shown in Sequence 6 in the sequence listing was inserted into the Xcm I restriction site of the pBCXUN vector to obtain a recombinant plasmid.

2. The method was the same as 2 in step 1 of Example 3.

3. The recombinant Agrobacterium obtained in step 2 was taken, and the Agrobacterium-mediated method was used to genetically transform the immature embryos of maize inbred line B73 to obtain T0 generation plants.

4. The TO generation plants were selfed, the seeds were harvested, and the seeds were cultivated into plants, that is, the T1 generation plants.

5. The T1 generation plants were identified by PCR, and transgenic plants were screened. The transgenic plants selected from the T1 generation plants were the T1 transgenic plants. A number of transgenic plants were selected from the T1 generation plants, three of which were named RI plant, R2 plant, and R3 plant.

PCR identification method: plant leaves were taken, genomic DNA was extracted therefrom, and a primer pair composed of F₃ and R3 was used for PCR amplification. If the 357 bp amplification product was obtained, and the PCR identification was positive, the plant was a transgenic plant. If no amplification product was obtained, and the PCR identification was negative, the plant was a non-transgenic plant.

F3:
(SEQ ID NO: 18)
GGTGGACGGCGAGGTCGCCG;
R3:
(SEQ ID NO: 19)
TCGGTGACGGGCAGGACCGG.

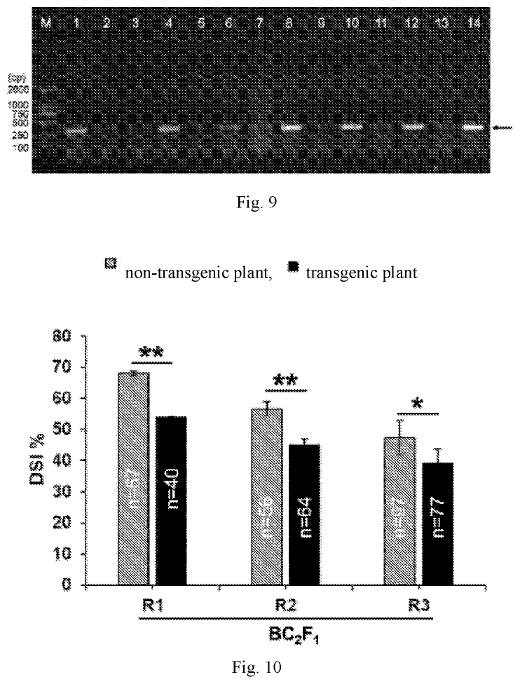

The PCR identification results of some plants are shown in FIG. 9. The arrow marks the target band. The leftmost lane is the molecular weight standard (M). Each of the remaining lanes corresponds to a plant (numbered 1 to 14).

II. Obtaining the Offspring Separated from Backcrossing

The PCR identification method was the same as 5 in step I.

The R1 plant (or R2 plant or R3 plant) as the male parent was crossed with the maize inbred line Q11 as the female parent. The seeds were harvested, and cultivated into plants, that is, the BC1F1 plants. The transgenic plants were identified and screened by PCR. The transgenic plant of the BC1F1 plants was used as the male parent to cross with the maize inbred line Q11 as the female parent. The seeds were harvested, and cultivated into plants, that is, the BC2F1 plants. The transgenic plants and non-transgenic plants were identified and screened by PCR.

### III. Identifying Plant Disease Resistance

### 1. Methods of Identification of Disease Resistance

The method was the same as 1 in step IV of Example 2.

2. Identifying the Disease Resistance of the Offspring Separated from Backcrossing (BC2F1 Plants)

Test materials: the transgenic plants and non-transgenic plants of the BC2F1 plants obtained with the R1 plant as the male parent as shown in step II, the transgenic plants and non-transgenic plants of the BC₂F₁ plants obtained with the R2 plant as the male parent as shown in step II, and the transgenic plants and non-transgenic plants of the BC₂F₁ plants obtained with the R3 plant as the male parent as shown in step II;

The test materials were identified for disease resistance according to the method in step 1. The disease index of the transgenic plants was significantly lower than that of the non-transgenic plants, and DSI decreased by 8.4-14.1%.

The results were shown in FIG. 10 (the gray bars are non-transgenic plants, and the black bars are transgenic plants. The numbers in the bar graph indicate the number of plants. *: P<0.05; **: P<0.01).

The above results indicate that backcrossing the ZmWAK-RLK gene into the Q11 genetic background can significantly improve the resistance of maize to gray leaf spot. At the same time, it also shows that the N-terminal segment of ZmWAK-RLK protein (the amino acids 1 to 324 of Sequence 1 in the sequence listing) plays an important role in the resistance to gray leaf spot of maize.

### INDUSTRIAL APPLICABILITY

The inventors of the present invention provide ZmWAK-RLK protein and its encoding gene. The experiments of overexpression of the transgene prove that the ZMWAK-RLK gene can increase the resistance of maize to gray leaf spot and significantly reduce the disease index of maize gray leaf spot. The present invention has great application value for the breeding of maize against gray leaf spot.

1-16. (canceled)

17. A maize plant comprising a recombinant nucleic acid encoding a protein comprising the amino acid sequence of:
(a1) SEQ ID NO: 1;
(a2) SEQ ID NO: 7;
(a3) SEQ ID NO: 1 or SEQ ID NO: 7 linked to a tag at an N-terminus or/and a C-terminus of the protein;
(a4) an amino acid sequence that differs from SEQ ID NO: 1 or SEQ ID NO: 7 by substituting and/or deleting and/or adding one or a plurality of amino acid residues of SEQ ID NO: 1 or SEQ ID NO: 7; or
(a5) an amino acid sequence having at least 90% sequence identity to SEQ ID NO: 1 or SEQ ID NO: 7, wherein the recombinant nucleic acid is located at a position other than the ZmWAK RLK genomic locus.

18. The maize plant of claim 17, wherein the recombinant nucleic acid comprises:
(b1) nucleotides 87 to 2084 in SEQ ID NO: 2;
(b2) SEQ ID NO: 2;
(b3) SEQ ID NO: 3;
(b4) SEQ ID NO: 6;
(b5) a DNA molecule having at least 90% sequence identity with (b1), or (b2), or (b3), or (b4); or
(b6) a DNA molecule that hybridizes to (b1), or (b2), or (b3), or (b4) under a stringent condition.

19. The maize plant of claim 17, wherein the recombinant nucleic acid is a transgene.

20. The maize plant of claim 17, wherein the recombinant nucleic acid is a genome fragment introduced by gene editing.

21. The maize plant of claim 17, wherein the maize plant has increased resistance to gray leaf spot disease as compared to an otherwise isogenic maize plant lacking the recombinant nucleic acid.

22. The maize plant of claim 17, wherein the maize plant has increased resistance to a disease caused by Cercospora zeina as compared to an otherwise isogenic maize plant lacking the recombinant nucleic acid.

23. A method of introducing a nucleic acid sequence for increasing resistance of a maize plant to a disease, the method comprising
providing a starting maize plant having susceptibility to disease, wherein the disease is gray leaf spot or a disease caused by Cercospora Zeina; and
introducing a nucleic acid sequence into the starting maize plant to thereby obtain a an altered maize plant, wherein the nucleic acid sequence encodes a protein comprising the amino acid sequence of:
(a1) SEQ ID NO: 1;
(a2) SEQ ID NO: 7;
(a3) SEQ ID NO: 1 or SEQ ID NO: 7 linked to a tag at an N-terminus or/and a C-terminus of the protein;
(a4) an amino acid sequence that differs from SEQ ID NO: 1 or SEQ ID NO: 7 by substituting and/or deleting and/or adding one or a plurality of amino acid residues of SEQ ID NO: 1 or SEQ ID NO: 7; or
(a5) an amino acid sequence having at least 90% sequence identity to SEQ ID NO: 1 or SEQ ID NO: 7.

24. The method of claim 23, wherein the nucleic acid sequence comprises:
(b1) nucleotides 87 to 2084 in SEQ ID NO: 2;
(b2) SEQ ID NO: 2; (b3) SEQ ID NO: 3;
(b4) SEQ ID NO: 6;
(b5) DNA having at least 90% sequence identity with (b1), or (b2), or (b3), or (b4); or
(b6) DNA that hybridizes to (b1), or (b2), or (b3), or (b4) under a stringent condition.

25. The method of claim 23, wherein the method comprises introducing the nucleic acid sequence as a transgene.

26. The method of claim 23, wherein the method comprises use of genome editing to introduce the nucleic acid sequence.

27. The method of claim 23, wherein the method comprises allele exchange to replace a resistant ZmWAK-RLK allele in the starting plant with the nucleic acid sequence.

28. The method of claim 26, wherein the method comprises allele exchange to replace a susceptible ZmWAK-RLK allele in the starting plant with the nucleic acid sequence.

29. The method of claim 23, wherein the method comprises introducing a genomic fragment comprising the nucleic acid sequence.

30. The method of claim 26, wherein the method comprises introducing a genomic fragment comprising the nucleic acid sequence.

31. The method of claim 26, wherein the method comprises modifying a susceptible ZmWAK-RLK allele in the starting plant to thereby introduce the nucleic acid sequence.

32. The method of claim 23, wherein the altered maize plant has increased resistance to the disease as compared to the starting maize plant.
